(19) United States
(12) Patent Application Publication
(10) Pub. No.: US 2021/0313029 A1
(43) Pub. Date: Oct. 7, 2021

(54) SYSTEM AND METHOD FOR PROMOTING A CHANGE IN CONSUMER SMOKING HABITS

(71) Applicant: Philip Morris Products S.A., Neuchatel (CH)

(72) Inventors: Robert EMMETT, Neuchatel (CH); Axel TEICHERT, Neuchatel (CH); Michel BESSANT, Neuchatel (CH)

(73) Assignee: Philip Morris Products S.A., Neuchatel (CH)

(21) Appl. No.: 17/270,199

(22) PCT Filed: Aug. 29, 2019
(86) PCT No.: PCT/EP2019/073154

§ 371 Date: Feb. 22, 2021

(30) Foreign Application Priority Data
Aug. 31, 2018 (EP) 18192105.7

(51) Int. Cl. G16H 15/00; G16H 50/70; G16H 10/60; G16H 40/67; G16H 10/40; G06F 16/26; A24F 40/60; A24F 40/65; G01N 33/49; A61B 5/00

(52) U.S. Cl. CPC G16H 15/00; G16H 50/70; G16H 10/60; G16H 40/67; G16H 10/40; A61B 5/742; A24F 40/60; A24F 40/65; G01N 33/49; A61B 5/4845; G06F 16/26

(57) ABSTRACT

A method of assisting smoking cessation is provided, including: measuring a level of a toxicant in a user; displaying the measured level of the toxicant to the user; providing the user with a heated tobacco product or an electronic smoking device; measuring a new level of the toxicant in the user after a period of use of the heated tobacco product or the electronic smoking device; and displaying the new level of the toxicant to the user. A toxicant testing station is also provided, including: a toxicant testing device to detect a level of a toxicant in a user; a processing device connected to the testing device; and a display device connected to the processing device, the processing device to provide to the display device a graphical representation of the level of toxicant detected by the testing device in comparison to an average level of toxicant within a population.

The present invention relates to a network of retail stores offering alternatives to conventional cigarettes and a method to help smokers of conventional cigarettes cease smoking or change to alternative smoking products.

Many people regularly smoke conventional cigarettes. Habitual smoking of conventional cigarettes can result in smokers taking in elevated levels of certain toxicants compared to non-smokers or smokers of alternative products, such as heated tobacco systems or other alternative such as e-cigarettes. It would be desirable to help smokers of conventional cigarettes change their habits by ceasing smoking or moving to less harmful alternatives.

However, persuading smokers to change their smoking habits can be difficult. Programmes for helping smokers cease smoking or change to alternative products are typically available only in clinical environments, such as doctors' surgeries. Smokers can be reluctant to visit such a clinical environment. Even within a clinical environment, it can be difficult to convey the benefits of stopping smoking or of using alternatives to conventional cigarettes, such as heated tobacco products, in a way that is convincing and personal.

For example, it would be desirable to be able to demonstrate to smokers the benefits of stopping smoking, in a relatively short time. If the smoker is not inclined to stop smoking altogether, it would be desirable to be able to demonstrate the benefits of smoking heated tobacco products, or other alternative products such as e-cigarettes, as an alternative to smoking conventional cigarettes, in a relatively short time. Such products, when used instead of conventional cigarettes, have the potential to reduce users' exposure to constituents known to be harmful. It would also be desirable to be able to capture data of user behaviour with heated tobacco products or e-cigarettes, and the benefits of heated tobacco products or e-cigarettes as compared to conventional combustible cigarettes, so that better substantiated information can be provided to customers and regulatory bodies. Finally, it would be desirable to be able to provide population level statistics relating to the use of alternatives to conventional cigarettes, such as heated tobacco products.

According to a first aspect of the present invention there is provided a network of retail stores offering alternatives to conventional cigarettes, such as heated tobacco devices, wherein the network of retail stores comprises two or more retail stores, each in a different location, and a central server configured to host a central database storing customer data, wherein each individual retail store is connected to the central server.

Each individual retail store in the network of retail stores comprises a toxicant testing station, the toxicant testing station comprises: at least one toxicant testing device for detecting a level of a toxicant in a customer; a data entry device configured to receive customer information; a processing device connected to the toxicant testing device and the data entry device, and configured to send data to, and receive data from, the central server, wherein the data comprises customer information data and detected toxicant level data; and a display device connected to the processing device, the processing device configured to provide to the display device a graphical representation of a comparison between the level of toxicant detected by the toxicant testing device and a previously detected level of toxicant for the customer or an average level of the toxicant within a population.

It is advantageous that the toxicant testing station is part of a retail store, and so in a retail environment, rather than being in a clinical testing environment. In a retail environment the smoker is a customer. In a clinical environment the smoker is a patient. The smoker being a customer rather than a patient is advantageous as the smoker will typically feel more comfortable and in control of the situation, and more likely to consider the benefits of stopping or changing from conventional smoking.

It is also more likely that a smoker will enter the retail store than a clinical setting. For example, a customer may enter a retail store before having decided that they want to change smoking habits. However, for a smoker to enter a clinical setting as a patient, the smoker needs to already have decided that they have a habit they want to change.

A retail environment can be made to feel more appealing to a smoker than a clinical environment and so a smoker may feel more inclined to drop into a retail store than they would a clinical environment. This will be particularly true if the retail store and/or testing station is visually appealing. For example, the testing station may be integrated in a stand or a booth inside the retail store in a way which is visually appealing. To further improve the appeal of the retail store and/or testing station well-known branding may be used to which the customer trusts and feels loyal to.

Advantageously, a coach may be present in each retail store who is specially educated with professional knowledge in helping customers cease smoking and/or change to using alternatives. The coach may explain the benefits of stopping or changing from smoking conventional cigarettes before asking the customer if they would like to perform the toxicant test. The coach may explain that smoking causes elevated toxicant levels.

Above average toxicant levels may be associated with risks of damage to the heart, lungs and respiratory system. The graphical representation of the level of toxicant detected by the toxicant testing device may advantageously be used to demonstrate to the customer the benefits of stopping smoking conventional cigarettes and/or smoking alternatives to such as heated tobacco products or e-cigarettes.

In some embodiments, the graphical representation compares the measured toxicant level with an average level of the toxicant within a population. This may be a population of non-smokers. A comparison showing a higher than average level of toxicant in a customer can be used to demonstrate to a customer that they are at increased risk of having a damaged heart, lungs and/or respiratory system. This may be used to persuade the customer stop smoking conventional cigarettes or instead change to smoking heated tobacco products, or another alternative product, such as an e-cigarette.

In some cases, the graphical representation compares detected toxicant level from a current test with a detected level of toxicant from a previous test result for the same customer. Advantageously, the detected toxicant level from the previous result is from a time when the customer was smoking conventional cigarettes and the current detected toxicant level is after the customer has stopped smoking conventional cigarettes or made the change to heated tobacco products instead. A reduction in the measured toxicant level compared to the toxicant level measured in the previous test can be used to persuade a customer to continue with their chosen change in smoking behaviour or smoking products. The comparison may also include the average toxicant level in a population, which may be a population of non-smokers. Showing the customer that not only is their toxicant level reduced compared to the previous test, but it is also substantially similar to the average toxicant level in the non-smoking population, may further persuade a customer to continue with their chosen change in smoking behaviour or smoking products.

The graphical representation of the toxicant level may advantageously have the effect of gamification of reduction of toxicant levels. Gamification may encourage customers of conventional cigarettes, reluctant to stop smoking conventional cigarettes and/or change to heated tobacco products, or other alternatives, such as e-cigarettes, and unpersuaded by the arguments alone, to make a permanent change to heated tobacco products. Gamification has the aim for the customer to maintain low toxicant level measurements of a period of time and over several testing sessions. This may incentivise the customer to refrain from smoking conventional cigarettes more effectively than simply making arguments highlighting the benefits.

The graphical representation may be a graph that quantitatively illustrates the measured toxicant level in the current test. A simpler graphical representation may be used showing “green”, “amber” or “red”, where green indicates improved or low toxicant levels, amber indicates unchanged or medium toxicant levels and red indicates worsened or high toxicant levels, either comparing toxicant level to a previous test or compared to the average general population. Customers may find the simpler, colour-coded, graphical interface more persuasive to make a permanent change from conventional cigarettes to heated tobacco products.

The toxicant testing device may be a breath testing device into which a customer exhales. The toxicant testing station may comprise a plurality of disposable mouthpieces for the toxicant testing device. The toxicant testing device may be a blood testing device. Both the breath testing device and the blood testing device advantageously may be simple to perform, comfortable and take only a short amount of time for the measurement. The toxicant testing device may be considered to be non-invasive.

The toxicant may be carbon monoxide. An example of a testing device that measures the carbon monoxide levels of a customer's exhaled breath is the BedFont Micro+™ available from Bedfont Scientific Ltd, Station Road, Harrietsham, Maidstone, Kent, ME17 1JA, ENGLAND. An example of a blood testing device that measures carbon monoxide is the Masimo Rainbow® CO-oximeter available from Masimo Corporation, Puits-Godet 10, 2000 Neuchatel, SWITZERLAND. Carboxyhemoglobin (COHb) levels in customer's blood may be measured to represent CO levels. Typically, the levels of measured CO in a consumer of conventional cigarettes will be approximately five times higher than the average within the population of non-smokers, although this depends on the frequency and quantity of conventional cigarettes the customer smokes in a day.

An example of a heated tobacco product is the IQOS Heatstick available from https://uk.iqos.com/shop. CO levels generated by the IQOS Heatstick are more than 98% reduced compared to conventional cigarettes. A habitual user of an IQOS Heatstick has determined CO levels substantially the same as the average CO levels within a population of non-smokers. When a customer changes from conventional cigarettes to heated tobacco products, such as the IQOS Heatstick, the levels of COHb in the blood stream rapidly decreases. COHb levels may be substantially reduced to levels comparable to the average non-smoker general population within one or two days of the customer making the change. A comparison showing a decrease of CO levels after changing from conventional cigarettes to a heated tobacco device, such the IQOS Heatstick, may then motivate the customer to stop smoking conventional cigarettes or to continue to smoke heated tobacco, or other alternatives, such as e-cigarettes, rather than conventional cigarettes.

It is advantageous that there are at least two individual retail stores located in a different locations to one another comprising the toxicant testing station. The customer does not then need to return to the same store to perform a further test and observe the change in the level of the toxicant. The measured toxicant levels are stored and retrieved from the same central server regardless of the individual store in which the user performs the test.

The central server may store detected toxicant levels for comparison with a detected toxicant level from a toxicant test carried out by the customer at a later time. The comparison may be between toxicant levels detected in two different individual retail stores in the network of retail stores.

By storing all data in a centralised server, the data of a large number of customers, received from a plurality of individual stores, can be analysed. This allows for population level statistics relating to the switching effect, i.e. the reduction in toxicant exposure after switching from conventional cigarettes to heated tobacco products, or other alternatives, such as e-cigarettes, or stopping smoking altogether. A machine learning technique may be used to analyse the data collected in the central database to build a reference database which can be used to cross-compare various heated tobacco products and any associated benefit relating to the reduction of toxicants typically measured, both compared to one another and compared to conventional cigarettes.

The toxicant testing station may comprise an interface for a customer's heated tobacco device. Usage data from the heated tobacco device may be downloaded through the interface. The display device may be configured to display usage data from the customer's electronic smoking device together with the level of toxicant detected.

The customer information may comprise information about one or more of the customer's: smoking history, occupation, lifestyle, home location, working location, medical conditions and diet. There may be a link between such environmental information about the customer and the toxicant level that is measured.

The processing device may be configured to generate an anonymous customer identifier for the customer. Identifying customer information may be stored together with the anonymous customer identifier in a first database and non-identifying customer information and detected toxicant levels may be stored together with the anonymous customer identifier in a second database. The anonymous customer identifier helps to uniquely identify the customer. Any customer information, including any toxicant level measurements, stored in the central server are stored alongside the anonymous customer identifier. This allows data relating to the customer from previous tests that may have been performed in stores different to the store the current test is being performed in, to be retrieved from the central server. By storing personal customer information, such as name and address, in a separate database to the sensing data and environmental data, the toxicant data may be used anonymously, so that the data of individual customers and whole populations of customers can be analysed in research without compromising data privacy. Such research may involve studying links between successive toxicant measurements and environmental data or studying overall population smoking behaviour.

According to a second aspect of the present invention there is provided a method of assisting smoking cessation, comprising:
measuring an initial level of a toxicant in a customer in a first retail store forming part of a network of at least two retail stores;
sending measured initial level of toxicant data together with a customer identifier to a database stored on a central server;
displaying the initial measured level of toxicant to the customer;
providing the customer with a heated tobacco product or electronic smoking device;
measuring a new level of the toxicant in the customer after a period of use of the heated tobacco product or electronic smoking device;
receiving the initial level of toxicant from the database; and displaying a comparison of the new level of toxicant and the initial level of toxicant to the customer.

The step of measuring a new level of the toxicant may be performed in a different retail store in the network of at least two retail stores to the first retail store.

The step of displaying may comprise displaying a comparison of the initial or new level of toxicant to an average level of the toxicant within a population.

Embodiments of the invention will now be described, by way of example only, with reference to the accompanying drawings, in which:

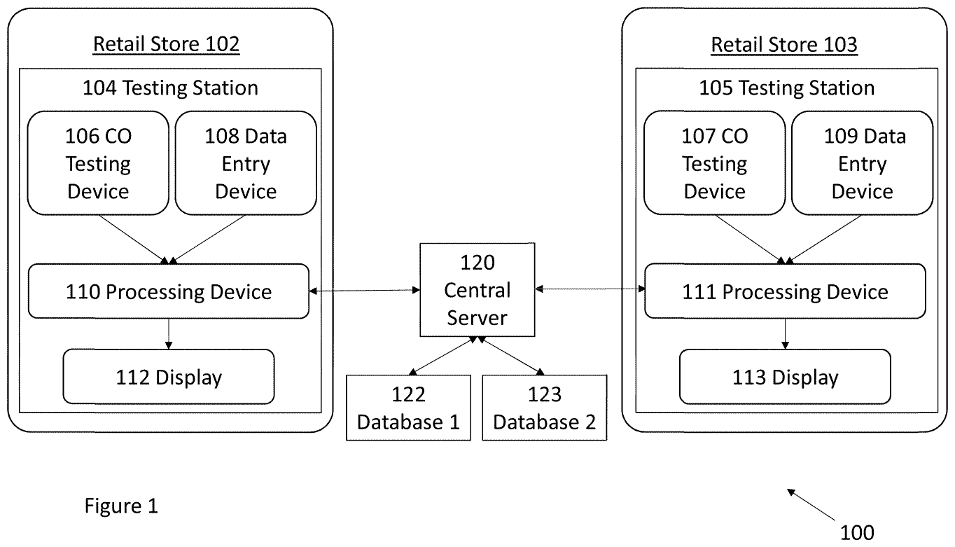

FIG. 1 is a schematic diagram showing a network of retail stores, in accordance with the invention, comprising two individual stores;

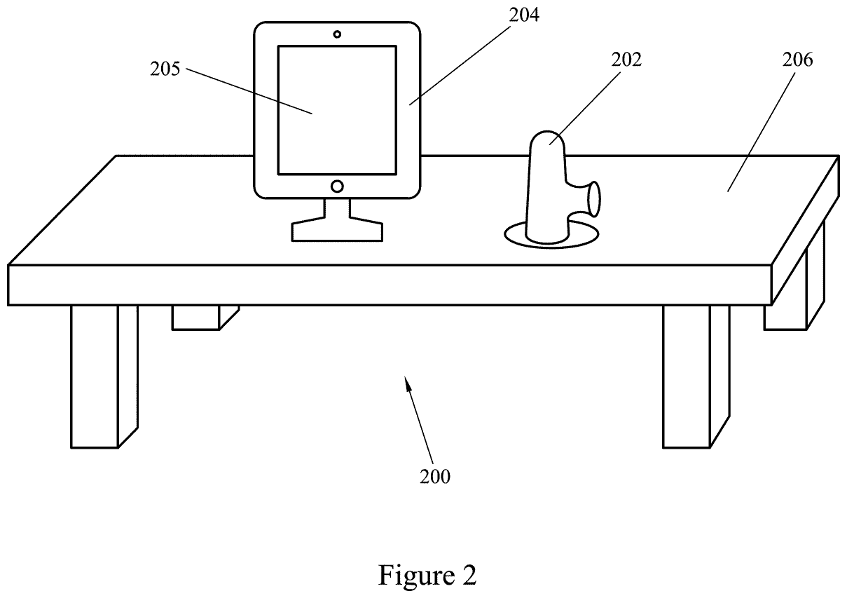

FIG. 2 is a schematic illustration of a testing station;

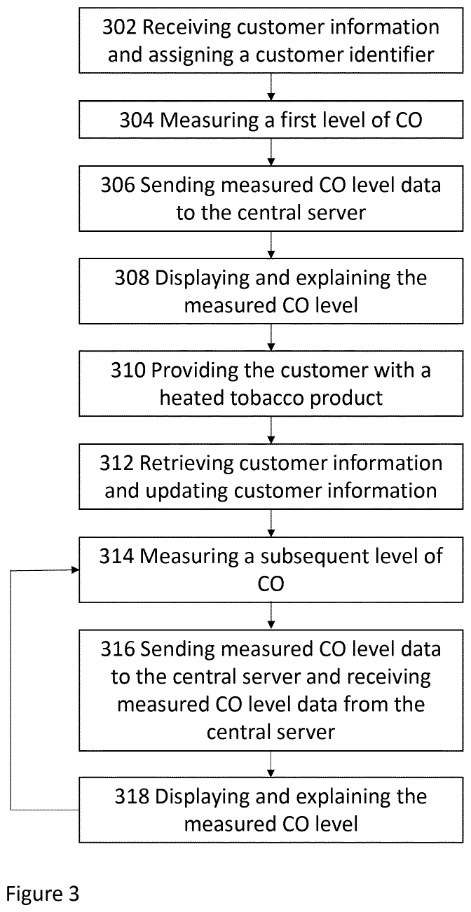

FIG. 3 is a flow chart of a method of assisting smoking cessation in accordance with the invention; and

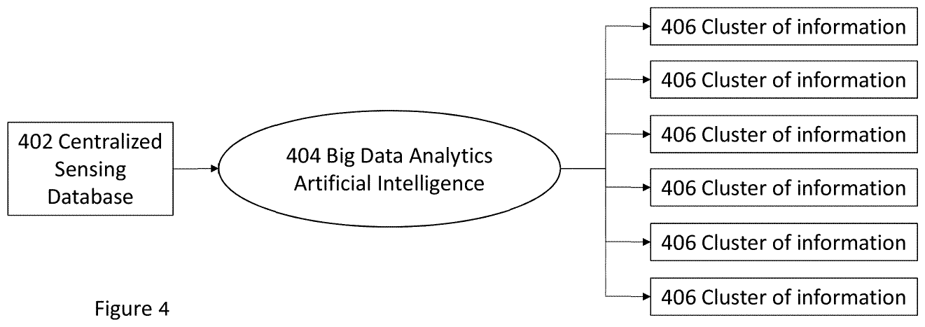

FIG. 4 is a schematic diagram showing the use of big volume data analytics and artificial intelligence to analyse one of the databases on the central server to form clusters of information representing population sub-groups.

A network of retail stores can be used to help a customer of those stores to cease the smoking of conventional cigarettes or, if the customer is not inclined to stop smoking conventional cigarettes completely, persuade the customer of the benefits of changing to alternatives, such as heated tobacco products. FIG. 1 is a schematic of such a network of retail stores 100. The network of retail stores 100 comprises two individual retail stores 102 and 103 and a single central server 120.

Each individual retail stores offers alternatives to conventional cigarettes, such as heated tobacco products. A mentor or store coach is present in each of the stores. The coach welcomes the customer and then, in an informal conservation, explains the benefits of stopping smoking conventional cigarettes, or, if the customer is not inclined to stop smoking conventional cigarettes, the benefits of switching from smoking conventional cigarettes to smoking of heated tobacco products as an alternative. The informal conversation, combined with the comforting and relaxing retail environment of the store, may help to persuade the customer to engage with the coach. The coach is then able to persuade the customer to perform a toxicant test, which in this embodiment is a carbon monoxide (CO) test. The results of the toxicant test can be used to demonstrate to a customer that they have elevated CO levels as a result of smoking conventional cigarettes compared to an average in a population of non-smokers. The coach then explains the negative effects of such levels of CO and tries to persuade the customer to stop smoking and/or switch to smoking heated tobacco products offered by the retail store.

A customer should have elevated levels of CO, as determined for instance in exhaled breath or by pulse oximetry (COHb) if they have smoked a conventional cigarettes less than four hours prior to testing. If it has been longer than four hours since the customer smoked a conventional cigarette, the coach may ask the customer to go out of the store and smoke such a product, before returning to perform the test. The coach will first ask the age of the customer to ensure they are older than the legal age for smoking. The coach may perform the test immediately before and after the customer smokes a conventional cigarette to demonstrate the contrast in CO levels, and so the link between smoking conventional cigarettes and elevated CO levels in the blood or breath.

The customer is invited to return to one of the stores in the network of retail stores to perform the CO test again at a later date. This subsequent test will reveal that the user has a lower level CO as a result of stopping smoking altogether or using heated tobacco products. This lower level of CO is much closer the average in the general non-smoker population. This encourages the customer to continue to not smoke conventional cigarettes or continue using heated tobacco products instead. By comparing CO levels to previous tests and the average in a population of non-smokers, CO levels are effectively gamified. Gamification encourages customers to maintain low CO levels, and so not return to smoking conventional cigarettes.

Retail store 102 comprises a testing station 104. The testing station is configured to perform the test to detect or measure the level of CO in the blood or breath of a customer. The testing station 104 comprises one or more CO testing devices 106 for performing the test to measure the level of CO, a data entry device 108 which can be used to input particular customer information, a processing device 110 and a display 112. The processing device 110 is connected to the CO testing device 106 and the data entry device 108 such that it can receive data from both. The processing device 110 is also connected to the central server 120. The processing device can transfer data to and from the central server. The processing device can manipulate data such that comparisons between different data sets can be made.

Retail store 103 comprises a similar testing station 105 which comprises similar features: a toxicant testing device 107, a data entry device 109, a processing device 111 and a display 113. The processing device 111 is connected to the central server 120.

It should be understood that FIG. 1 shows only two stores for simplicity. However, the network of retail stores may comprise more than two individual retail stores, where each of the individual retail stores comprises a similar testing station and processing device as described above. Data can be sent and received between the central server 120 and the respective processing device in the testing stations in each of the retail stores.

FIG. 2 is an example of a testing station that could be placed in either of the retail stores 102, 103 of FIG. 1. The testing station 200 should be placed in an area of the retail store that is quiet and private in order to ensure that the customer has a relaxing experience and is able to have a personal discussion with the coach. The testing station 200 comprises a testing device 202. In this embodiment, the testing device is a BedFont Micro+™ device available from https://www.intermedicaldirect.co.uk which is used to perform a test for measuring the CO levels in blood. In order to perform a test the customer simply breathes normally into the device. The device measures the number of CO molecules in ppm in the customer's breath. The device itself comprises a display which can show the results of the test, either as percentage or by turning the percentage into a red, amber, green output. However, the testing device 202 is connected to the processing device 204 which is programmed with software to receive results from the testing device 202. The processing device 204 can calculate the percentage of red blood cells that carry CO instead of oxygen for the number of CO molecules in ppm in the customers breath if necessary. The connection between the testing device 202 and the processing device 204 may be through a wired or wireless connection between the testing device and the processing device.

In FIG. 2 the processing device is a tablet computer and comprises a display 205. The display comprises a touch screen and also acts as a data entry device. The data entry device 108, the processing device 110 and the display 112, are all incorporated in the tablet computer 204 of FIG. 2. The tablet computer is connected to the central server, as shown in FIG. 1. This connection is through the internet, either via a wired or wireless connection, or both. The testing station comprises a table 206 on which the testing device and the display are placed.

FIG. 2 only shows one embodiment of the testing station 202. However, numerous other embodiments are possible. The data processing device may be positioned flat and in the table 206. Alternatively, the data processing device may be configured such that the display 205 is pointed toward the coach and a second display is pointed toward the customer. Preferably, the testing stations have a substantially uniform appearance across all the individual stores in the network of stores. This ensures that a customer visiting any of the stores finds the testing station familiar.

FIG. 3 shows a flow chart of a method of assisting smoking cessation for a customer using the network of retail stores. Prior to step 302 a customer enters a retail store and is met by one of the store coaches. The store coach explains to the customer the benefits of smoking heated tobacco products instead of conventional cigarettes. The store coach then persuades the customer to take part in a first test to measure a CO level.

In step 302 the coach records customer information. The data entry device is used to input any desirable additional information relating to the customer. This may be information to identify the customer, as well as data such as the customer's smoking history, occupation, lifestyle, home location, working location, medical conditions and diet is also input into the data entry device. The processing device assigns a unique identifier number or character string to the customer, that is anonymous.

The test is performed in step 304. The testing device 202 comprises a replaceable mouthpiece. For each testing session a new mouthpiece is attached to the testing device. At step 304 a new mouthpiece is fitted to the testing device 202 and the test is performed. This step is performed by the customer breathing into the testing device 202. A level of CO is measured and recorded. The testing device measures the number of CO molecules in ppm in the customers breath and can calculate the percentage of red blood cells that carry CO instead of oxygen. The measurement can be performed quickly, using a single breath into the device. The test is easy to perform and is not stressful. Data from the testing device is transferred to the processing device.

At step 306 the processing device sends and/or receives data relating to CO level measurements to the central server. The customer information and sensing data is transferred to and stored on the central server. The central server comprises, or is connected to, two databases 122, 123, as shown in FIG. 1. In both databases, information is stored together with the customer's unique identifier number. This unique identifier number can then be used to retrieve data relating to that customer at a later time. Any personal data that could be used to identify the customer, such as age and address, is stored in the first database 122. Measured toxicant level data is stored anonymously in a second database 123.

At step 306, the processing device may also receive data relating to average CO levels in a population of non-smokers, as calculated from data in the sensing database. Note that in some embodiments the receiving of this data from the central server may be performed periodically, independent of any test being performed, or may be performed as part of step 302.

At step 308 the results of the test are displayed to the customer on the display 205. The result is based on the percentage of blood comprising carboxyhaemoglobin and this percentage is inferred from the measured ppm of CO. Calculations relating to the inference are performed by the processing device. Alternatively, the calculations may be performed by the testing device before the data is transferred to the processing device.

The results are displayed graphically on the display 205. In some embodiments this is in the form of a graph showing the percentage of carboxyhaemoglobin in the blood of the consumer. Plotted on the graph are current result of the test and an average value for a population of non-smokers. The store coach explains the results to the customer and educates about its meaning. Typically, the CO levels in a smoker will be higher (possibly five times higher) than in the population of non-smokers. The store coach can use this to persuade the customer to stop smoking conventional cigarettes and/or try a heated tobacco product, which the customer is provided with at step 310.

In another embodiment the graphical representation may simply be an indicator showing “green”, “amber” or “red”. For the first test, the indicator represents a comparison between measured CO level and the CO level of the average non-smoker general population. Green indicates low CO levels, i.e. better than or substantially equal to, the average non-smoker general population. Green therefore shows that the CO level is not elevated. Amber indicates a CO level between the CO level of the average non-smoker general population and four times that value. Amber therefore shows that the CO level is elevated, but not as high as would be had the customer very recently smoked a conventional cigarette. Red indicates a CO level higher than, or substantially equal to, five times the average CO level in the average non-smoker general population. Red therefore indicates an elevated CO level, and particularly an elevated CO level that is representative of a frequent smoker. It would be expected that most customers that are smokers would receive a red result in their first test. The coach may also find it more persuasive to show the customer such a red indicator rather than explaining a graph.

After step 310 the customer is expected to stop smoking conventional cigarettes or use the heated tobacco product instead. After a period of time, which is typically a few days or a week, the customer visits a retail store in the network of retail stores in order to perform the CO test again. This may be the same store as the store where the customer performed the previous test, or a different store. In step 312 the customer's information is retrieved and may be updated if there are any changes or additions.

At step 314 the store coach in the retail store will help the customer to perform another CO test to measure CO levels, including fitting a fresh mouthpiece to the testing device 202. The store coach will also have a discussion with customer. This discussion is to confirm whether the customer has refrained from smoking conventional cigarettes prior to visiting the store.

At step 316 the processing device sends data relating to the latest CO level measurements to the central server. The results from the initial test performed in step 304 are also downloaded to the processing device. It does not matter whether the user visited different retail stores at steps 302 and 310 because all the data is stored in centrally accessible databases and can be accessed by the data processing device in each store. Therefore, the same customer can test their CO level in any store seamlessly and regardless of geographical location. The result of the test performed at step 310 is transferred to the central database by the data processing device. In this case, there exists at least one historical test for the customer stored on the central database, which is the result from step 304. The data processing device can receive this test result using the unique identifier number for the customer.

At step 318 the results of the test are displayed to the customer on the display 205. The store coach explains the results to the customer. Again, the results are shown in a graphical representation, either as a graph or as a simplified indicator. If shown as a graph, historic test results are plotted on the graph, as well as the current test result. This allows the store coach to highlight the comparison in CO levels over time as a result of changing in smoking behaviour or product use. If the customer has not smoked conventional cigarettes since step 304, then CO levels should have decreased. Again, the CO level of the average non-smoker general population is also plotted for comparison. The result from a test performed at 314 should be much closer to this average. The coach will use these comparisons to highlight to the customer the benefits of stopping smoking conventional cigarettes or the continued use of heated tobacco products instead, in view of the reduced toxicant levels.

In the case of the simpler graphical representation, again an indicator is used to show whether the CO levels are “green”, “amber” or “red” compared to the average in the average non-smoker general population. When there are historic tests a further indicator is also shown. This indicator shows whether the customer has improved CO levels compared to previous tests. This may be in the form of an up or down arrow or a further colour-coded indicator. The further colour-coded indicator shows green if the CO level is lower, i.e. improved, compared to previous tests, amber if the CO level is substantially equal to previous tests and red if the CO level is higher, i.e. worsened, since the previous test. The colour-coded indicators may help gamify the testing. Customers would be encouraged to maintain green coloured indicators and be disappointed should the indicators ever be red.

The customer may continue to periodically return to retail stores in the network of retail stores for CO testing. Steps 314 to 318 may be repeated for each subsequent testing session. The customer may find the testing process gamifies ceasing smoking. In this case, the customer will wish to maintain the CO levels to be low and substantially equal to the average for a population of non-smokers. This incentivises the customer not to revert back to conventional cigarettes.

The testing device does not have to be a device for measuring CO levels in exhaled breath. For example, in other embodiments, the testing device may be Masimo Rainbow @Pulse CO-oximeter. This is another test for measuring CO levels in a customer. This device measures CO in the blood. Any CO testing device that is clinically validated may be used.

Furthermore, other toxicants may be tested instead of or in addition to CO. Any toxicant whose level in the body of a smoker is elevated due to smoking cigarettes but which reduces as a result of cessation of smoking of conventional cigarettes may be used, particularly if a quick and test can be used to measure the level of toxicants taken up. Such toxicants include, for instance, nitric oxide, nitrogen oxides, benzene acetaldehyde, acrolein, acetone, isoprene, formaldehyde, toluene or hydrogen cyanid.

As previously described, the central server comprises two databases 122,123. The first database 122 contains any personal information about the customer that may be protected by data protection laws. The second database 123 contains non-identifying customer information and detected toxin levels. Any information in either of the databases is stored against the anonymous customer identification number. Therefore, the data stored in the second database 123 is completely anonymous. Prior to collecting data from any customer, the store coach will seek that customer's permission. Data is not used for advertising purposes but to allow scientific research to be undertaken.

The data of both individuals and larger population stored in the database can be analysed and evaluated in research studies without compromising data privacy. FIG. 4 shows a schematic diagram of how a sensing database 402 (equivalent to database 123 in FIG. 1) can be analysed using big data analytics methods and artificial intelligence 404. These methods can split the data into clusters of information 406 as desired. Each of the clusters of information may represent the behaviour of a population relating to links between, for example, how environmental impacts, changing of smoking behaviour, smoking behaviour of heated tobacco products, other health issues, or lifestyle, of a population may affect toxin levels. Comparison against non-smoker populations can also be made.

1.-13. (canceled)

14. A method of assisting smoking cessation, comprising:
measuring a level of a toxicant in a user;
displaying the measured level of the toxicant to the user;
providing the user with a heated tobacco product or an electronic smoking device;
measuring a new level of the toxicant in the user after a period of use of the heated tobacco product or the electronic smoking device; and
displaying the new level of the toxicant to the user.

15. The method of assisting smoking cessation according to claim 14, wherein the step of displaying the new level of the toxicant to the user comprises displaying a comparison of the new level of the toxicant with a previously measured level of the toxicant.

16. The method of assisting smoking cessation according to claim 14, wherein either the step of displaying the level of the toxicant or of displaying the new level of the toxicant comprises displaying a comparison of a respective level of the toxicant to an average level of the toxicant within a population.

17. The method of assisting smoking cessation according to claim 14,
further comprising at least one of sending or receiving data to/from a database stored on a central server,
wherein the data comprises data relating to at least one of the measured level of the toxicant and the new measured level of the toxicant.

18. The method of assisting smoking cessation according to claim 17, wherein the data further comprises user information including one or more of smoking history, occupation, lifestyle, home location, working location, medical conditions, and diet.

19. The method of assisting smoking cessation according to claim 18, wherein, when the method comprises sending the data to the database stored on the central server, the method further comprises:
storing identifying user information in a first database stored on the central server together with an anonymous user identifier that has been generated for the user; and
storing non-identifying user information and detected toxicant levels together with the anonymous user identifier that has been generated for the user in a second database on the central server.

20. The method of assisting smoking cessation according to claim 14, further comprising a step of downloading usage data from the heated tobacco product or the electronic smoking device and displaying the downloaded usage data together with the measured level of the toxicant.

21. The method of assisting smoking cessation according to claim 14,
wherein the step of displaying the measured level of the toxicant comprises displaying a graphical representation of the measured level of the toxicant, and
wherein the step of displaying the new level of the toxicant comprises displaying a graphical representation of the new level of the toxicant.

22. A toxicant testing station, comprising:
a toxicant testing device configured to detect a level of a toxicant in a user;
a processing device connected to the toxicant testing device; and
a display device connected to the processing device,
wherein the processing device is configured to provide to the display device a graphical representation of the level of the toxicant detected by the toxicant testing device in comparison to an average level of the toxicant within a population.

23. The toxicant testing station according to claim 22,
wherein the toxicant testing device is a breath testing device into which a user exhales, and
wherein the toxicant testing station further comprises a plurality of disposable mouthpieces for the toxicant testing device.

24. The toxicant testing station according to claim 22, wherein the toxicant testing device is a blood testing device.

25. The toxicant testing station according to claim 22, wherein the toxicant is carbon monoxide.

26. The toxicant testing station according to claim 22,
further comprising a data entry device configured to receive user information including one or more of smoking history, occupation, lifestyle, home location, working location, medical conditions, and diet,
wherein the processing device is further configured to send or receive data to/from a central server, the central server being configured to host a central database storing user data, the user data relating to toxicant level and the user information.

27. The toxicant testing station according to claim 22,
further comprising an interface for an electronic smoking device,
wherein the display device is configured to display usage data from the electronic smoking device together with the level of the toxicant detected.

28. The toxicant testing station according to claim 22, wherein the graphical representation is a color-coded representation in which different colors indicate whether the toxicant level detected by the toxicant testing device compared to an average level of the toxicant within the average population is: a) low, b) medium, or c) high.
